(19) United States
(12) Patent Application Publication
(10) Pub. No.: US 2024/0125753 A1
(43) Pub. Date: Apr. 18, 2024

(54) COMPOSITION AND METHOD FOR MEASURING CALCIUM HARDNESS IN PROCESS WATER

(71) Applicant: Ecolab USA Inc., Saint Paul, MN (US)

(72) Inventors: Hui Li, Bolingbrook, IL (US); Bingzhi Chen, Naperville, IL (US); Ashish Dhawan, Aurora, IL (US)

(21) Appl. No.: 18/484,097

(22) Filed: Oct. 10, 2023

Related U.S. Application Data
(60) Provisional application No. 63/414,632, filed on Oct. 10, 2022.
(60) Provisional application No. 63/477,444, filed on Dec. 28, 2022.

(51) Int. Cl. G01N 33/18; C02F 1/68; G01N 21/64

(52) U.S. Cl. CPC G01N 33/1853; C02F 1/68; G01N 21/6428; C02F 2209/06; G01N 2021/6439

(57) ABSTRACT

An indicator composition can be used to measure calcium hardness within a sample of water from a process water system. The indicator composition can include a calcium indicator, a range extender, and a buffer. The indicator composition can accurately measure calcium concentrations within the sample of water at low, moderate, and high calcium levels, including at calcium concentrations of greater than 50 ppm. The indicator composition can measure calcium hardness without the use of masking agents at a pH of less than about 7.

### RELATED APPLICATION

This application claims priority to U.S. Provisional Application No. 63/414,632, filed Oct. 10, 2022, and U.S. Provisional Application No. 63/477,444, filed Dec. 28, 2022, both entitled “Composition and Method for Measuring Calcium Hardness in Process Water,” the contents of which are hereby incorporated by reference in their entireties.

### TECHNICAL FIELD

This disclosure relates to compositions, methods, and systems of measuring calcium hardness in process waters, including, but not limited to, boiler water, cooling water, and wastewater systems.

### BACKGROUND

In water treatment industries, calcium is one of the major ions that forms scale on the water systems. Because calcium ions are naturally present in water, water treatment systems, such as, but not limited to, boiler water, cooling water, and waste water systems are faced with challenges involving the formation of scale on surfaces, especially on surfaces with elevated temperatures. Scale generally forms hard and adherent layers, causing processing challenges due to the retardation of heat transfer. Furthermore, scale within transfer pipes can reduce water flow, which requires higher pressure head and can accelerate additional fouling. While scale formation generally requires a temperature gradient, scale formation can still occur without a temperature differential under some conditions.

Common calcium scales found in water systems include calcium carbonate, calcium phosphate, and calcium sulfate. Generally, scale formation occurs when water containing dissolved mineral becomes supersaturated as the solubility of the mineral is exceeded. An induction period may be present, which is defined as the time required from the point of supersaturation to the initial formation of small particles of mineral scale. Crystallization of scale will typically occur if some degree of supersaturation exists, however, without the presence of some solid sites, a supersaturated solution will not form scale. As a result, water treatment systems are highly susceptible to scaling.

As scale grows, water flow rate decreases and pumping back-pressure increases. Because scale has lower thermal conductivity than metal heat exchanger surfaces, the presence of scale formation causes reduced heat transfer. As a result, decreased plant efficiency, reduced productivity, schedule delays, increase energy cost, loss of production from downtime for maintenance, and increased costs for equipment repair and replacement can occur. These effects directly lead to higher operating costs and decreased profitability. As the degree of supersaturation is a prime factor for controlling the deposition process, the concentrations of scale-causing minerals, such as calcium, should be monitored regularly to prevent scaling, thereby extending the lifespan of equipment and reducing the use of excess water, energy, and water treatment chemicals.

There are currently ways to measure calcium, including options to measure calcium in real time to provide more insight as well as possibilities to control scale stress to the equipment. Online methods of measuring calcium hardness in water include the ion-selective electrode (ISE), fluorescence, titration, and colorimetric methods. While colorimetric methods provide a convenient option for measuring calcium, accuracy is often compromised as many components within the water systems may interfere with calcium measurement. Further, in many process water systems, the calcium concentration in the water can reach as high as 1000 ppm (as CaCO₃) or more. However, dyes traditionally used to measure calcium, such as, for example, chlorophosphonazo-III used in Hach Method 8374, can only measure trace amounts of calcium up to 1 ppm (as CaCO₃).

Additional calcium indicators have been used, however, many are not thermally stable in liquid form. For example, U.S. Publication 2015/0198540 discloses methods of measuring calcium hardness in water using orthocresolphthalein (OCPC). However, OCPC was found to be thermally stable in liquid form for only two weeks, which is impractical for field and lab applications directed to process water systems. As such, there remains a need for additional compositions, methods, and systems, that can accurately measure calcium concentrations in process waters.

### SUMMARY

In general, this disclosure is directed to compositions, methods, and systems for the detection, identification, and measurement of calcium hardness in a sample of water, i.e., water from a process water system. An indicator composition is provided and can beneficially measure calcium concentrations at ranges much higher than current methods. The methods disclosed herein utilize a colorimetric method and the indicator composition to measure the concentration of calcium within the sample of water in a range of up to 1400 ppm or greater.

The indicator compositions of the disclosure are formulated to be able to measure moderate to high levels of calcium in a source of water. The indicator composition includes a weak ligand as a range extender to compete with the calcium indicator in such a way that the majority of the calcium ions within the source of water are complexed with the weak ligand. A small amount of remaining calcium ions may then react with the calcium indicator. Beneficially, the measurement range of the calcium indicator is extended. Further, a buffer may be included to maintain a pH at a level suitable for the calcium indicator to selectively chelate to calcium ions. This provides the benefit of not requiring additional masking agents to prevent the calcium indicator from chelating to other ions present in the source of water, such as magnesium.

In measuring the concentration of calcium within a water source, the absorbance, transmittance, or reflectance of a sample solution containing the water source and indicator composition can be obtained at a certain wavelength or a wavelength range. After the absorbance, transmittance, or reflectance is obtained, a linear calibration curve can be formed. The calibration curves using the indicator compositions of the disclosure can provide linear calibration curves for determining the concentration of calcium within the water source.

The indicator composition can exhibit excellent dimensional stability over a range of storage conditions, including comparatively high temperature conditions, for the duration of an expected storage period. In comparison to current indicators, the indicator composition of the disclosure can provide stability for a period of greater than two weeks. In some cases, the indicator composition may remain stable for a period of at least one month, or at least one year.

In one example, a method of measuring calcium hardness is described. The method includes extracting a sample of water containing calcium from a process water system and adding an indicator composition to the sample of water to form an optical analysis solution. The example specifies that the indicator composition can include a calcium indicator, a range extender, and a buffer. The method further includes a step of optically analyzing the optical analysis solution and determining therefrom a concentration of the calcium in the sample of water. As a result of determining the concentration of calcium in the sample of water, the addition of a calcium control agent to the process water system can be controlled or adjusted based on the determined concentration of calcium in the sample of water.

In another example, a system for measuring calcium is described. The system includes an optical sensor configured to receive a sample of water containing an unknown concentration of calcium from a process water system. The indicator composition may comprise a calcium indicator, a range extender, and a buffer. The system further includes at least one pump that is fluidly coupled to the indicator composition. Further, a controller is communicatively coupled to the optical sensor and the pump. The controller is configured to carry out a number of functions, including, controlling the pump to generate an optical analysis solution by at least combining the sample of water with the indicator composition, and controlling the optical sensor to optically analyze the optical analysis solution. Furthermore, the controller may be configured to determine a concentration of calcium in the sample of water based on the optical analysis.

In another example, an indicator composition for measuring a concentration of calcium is described. The indicator composition includes a calcium indicator, a range extender, and a buffer, wherein the indicator composition has a use pH less than 7.0. The example specifies that the indicator composition includes the calcium indicator in an amount of less than 0.5 wt %, the range extender in an amount of from about 2 wt % to about 10 wt %, and the buffer in an amount of from about 0.1 wt % to about 5 wt %. The composition may be provided as a solid composition or may be diluted with water to form a use solution prior to being added to a sample of water containing calcium from a process water system.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a graph illustrating the absorbance spectrum of the complex of calcium and chlorophosphonazo III. The graph provides the absorbance values at a wavelength of between 300 nm and 750 nm.

FIG. 2 is a graph illustrating the calibration curve of a reagent mixture of an indicator composition at room temperature. The calibration curve is shown as the ppm concentration of calcium as CaCO3 versus an absorbance ration at 668 nm versus 521 nm.

FIG. 3 is a graph illustrating the thermal stability of a reagent mixture measured at a wavelength of 668 nm and at a temperature of 120° F. over a period of 40 weeks.

FIG. 4 is a graph illustrating the thermal stability of a reagent mixture measured at a wavelength of 521 nm and at a temperature of 120° F. over a period of 40 weeks.

Various embodiments will be described in detail with reference to the figures. Reference to various embodiments does not limit the scope of the disclosure. Figures represented herein are not limitations to the various embodiments according to the disclosure and are presented for exemplary illustration.

### DETAILED DESCRIPTION

The embodiments of this disclosure are not limited to particular compositions, methods, and systems, which can vary and are understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

The methods, compositions, and systems of the present disclosure may comprise, consist essentially of, or consist of the components and ingredients of the present disclosure as well as other ingredients described herein. As used herein, “consisting essentially of” means that the methods, systems, and compositions may include additional steps, components or ingredients, but only if the additional steps, components or ingredients do not materially alter the basic and novel characteristics of the claimed methods, systems, apparatuses, and compositions.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

As used herein, the term “about” modifying, for example, the quantity of an ingredient in a composition, concentration, and like values, and ranges thereof, employed in describing embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture and amounts that differ due to mixing or processing a formulation with a particular initial concentration or mixture.

As used herein, the term “absorbance” means a quantitative measure expressed as a logarithmic ratio between the radiation falling upon a material and the radiation transmitted through a material according to the equation:

$${A}_{λ} = - {log}_{1} o ( \frac{{I}_{1}}{{I}_{0}} )$$

where Aλ is the absorbance at a certain wavelength of light (λ, I₁ is the intensity of the radiation (light) that has passed through the material (transmitted radiation), and I₀ the intensity of the radiation before it passes through the material (incident radiation). The amount of light transmitted through a material diminishes exponentially as it travels through the material. Since the absorbance of a sample is measured as a logarithm, it is directly proportional to the thickness of the sample and to the concentration of the absorbing material in the sample. Although absorbance is properly unitless, it is often reported in “Absorbance Units” or AU. Any real measuring instrument has a limited range within which it can accurately measure absorbance. An instrument must be calibrated and checked against known standards if the readings are to be trusted. Many instruments will become non-linear (fail to follow the Beer-Lambert law) starting at approximately 2 AU (˜1% Transmission). The theoretical best accuracy for most commercially available instruments is in the range near 1 AU. The path length or concentration should then, when possible, be adjusted to achieve readings near this range. Further meanings of the term are described in the Compendium of Chemical Terminology. 2nd ed. (the “Gold Book”), Published by IUPAC, (1997).

As used herein, the term “water” for treatment according to the disclosure includes a variety of sources, such as freshwater, pond water, sea water, salt water or brine source, recycled water, or the like. The term water is also understood to optionally include both fresh and recycled water sources, as well as any combination of waters for treatment according to the compositions of the disclosure. In some embodiments, recycled water refers to a mixture of water that comprises both water recycled from previous use (e.g., previous cycles of thermal exchange as a heat transfer medium) and water that has not been previously used (e.g., in cycles of thermal exchange as a heat transfer medium), such as, e.g., fresh water, pond water, sea water, etc.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used herein, “percent,” “%,” and the like are intended to be synonymous with “weight percent,” “wt %,” etc.

The present disclosure generally relates to calcium indicator compositions that are used to detect and measure calcium concentration in aqueous systems to ultimately control the formation of scale and corrosion deposits in the aqueous system. The aqueous system to be treated with the calcium indicator composition may typically be a cooling water system that supplies water to one or more processes in which thermal energy from a comparatively hot process stream is transferred to a comparatively cool water stream via a divided heat exchange surface. In some implementations, a indicator composition according to the disclosure can be used in an open circulating cooling water system, such as an open circulating cooling water system that includes one or more cooling towers that cool water via evaporative cooling. In other implementations, the aqueous system to be treated with the calcium indicator composition may be a wastewater treatment system, boiler water system, or other process water system.

In certain implementations, the indicator composition may generally include a calcium indicator, a range extender, and a buffer. The range extender may be provided as a weak ligand to compete with the calcium indicator for calcium ions within a water sample. In some aspects, this competition results in the majority of calcium ions within the water sample to be complexed with the range extender. This allows for a small amount of calcium ions to react with the calcium indicator to form a resultant compound that changes the light absorption of the calcium indicator. The relationship between the calcium indicator, the range extender, and the calcium ions within the water sample allow for the detection of a wide range of calcium concentrations in the water sample. In further aspects, the calcium indicator may be more selective to calcium ions over other ions within the water sample at a pH of less than 7, including at a pH of less than 6. For purposes of providing more accurate compositions in measuring the concentration calcium, a buffer may be provided to maintain pH in the range of less than 7. In further embodiments, the buffer may be provided to maintain pH in the range of less than 6. In some aspects, the maintenance of a pH level less than 7 absolves the need for additional masking agents to mitigate interference from other ions in the water sample, such as magnesium.

The indicator composition according to the disclosure includes at least one calcium indicator. In some aspects, calcium indicators are substances which show a change in a fluorescent characteristic upon binding with calcium. In certain implementations, the calcium indicator may include dyes such as, but not limited to, chlorophosphonazo III (CPA III), chlorophosphonazo I (CPA I), orthocresolphthalein (CPC), orthocresolphthalein (OCPC), methylthymol blue, arsenazo III, xylidyl blue, and Eriochrome Black T. In certain embodiments, the calcium indicator comprises CPA III. In some aspects, CPA III is selective to calcium ions over other ions in the pH range of less than 7. The calcium indicator may be present within the indicator composition in an amount of from about 0.001 wt % to about 1 wt %, from about 0.005 wt % to about 0.9 wt %, from about 0.01 wt % to about 0.8 wt %, from about 0.02 wt % to about 0.5 wt %. In further embodiments, the indicator is present within the indicator composition in an amount of less than 1 wt %, less than 0.9 wt %, less than 0.8 wt %, less than 0.7 wt %, less than 0.6 wt %, or less than 0.5 wt %.

The range extender of the indicator composition may comprise at least one of a polycarboxylic acid, an acrylate-based polymer, a polymaleic anhydride (PMA), a poly epoxy succinic acid (PESA), a maleic anhydride and sulfonated styrene copolymer (MA/SS), a polyaspartic acid, a chelating agent, or salts thereof. In some embodiments, the range extender comprises a polycarboxylic acid selected from the group consisting of citric acid, 1,2,3-propanetricarboxylic acid, saccharic acid, salts thereof, and combinations thereof. In some aspects, the range extender comprises an alkali metal citrate, such as sodium citrate. In some embodiments, the range extender comprises a polymer selected from the group consisting of a polyacrylate (PAA), a polymaleic anhydride (PMA), a poly epoxy succinic acid (PESA), a poly(acrylate-co-acrylamide) copolymer (AA/AM), an acrylic acid and hydroxypropylacrylate copolymer (AA/HPA), an acrylic acid and 2-acrylamido-2-methyl propane sulfonate copolymer (AA/AMPS), a maleic anhydride and sulfonated styrene copolymer (MA/SS), an acrylic acid/acrylamide/tertiary butyl acrylamide copolymer (AA/AM/t-BAM), an acrylic acid/2-acrylamido-2-methyl propane sulfonate/tertiary butyl acrylamide (AA/AMPS/t-BAM), an acrylic acid/sulfonated styrene/2-acrylamido-2-methyl propane sulfonate (AA/SS/AMPS), an acrylic acid/acrylamide/aminomethyl sulfonate copolymer (AA/AM/AMS), polyaspartic acid, salts thereof, and combinations thereof. In further embodiments, the range extender comprises a chelating agent selected from the group consisting of L-glutamic acid N—N-diacetic acid tetrasodium salt (GLDA), iminodisuccinic acid ((N-1,2-dicarboxyethyl)-D,L-aspartate acid), ethylenediamine-N,N′-disuccinic acid (EDDS), methylglycinediacetic acid (MGDA), ethyleneglycol-O,O′-bis(2-aminoethyl)-N,N, N′,N′-tetraacetic acid (EGTA), ethylenediamine-tetraacetic acid disodium salt (EDTA), ODS (oxygen disuccinic acid), salts thereof, and combinations thereof.

The range extender may be present within the indicator composition in an amount of from about 0.1 wt % to about 15 wt %, from about 0.1 wt % to about 10 wt %, 0.2 wt % to about 9 wt %, or from about 0.3 wt % to about 8 wt %. In some implementations, the range extender may be present within the indicator composition in an amount of less than 15 wt %, less than 12 wt %, less than 10 wt %, less than 9 wt %, or less than 8 wt %.

The indicator composition according to the disclosure can be pH-controlled, e.g., to provide a resultant solution upon dissolution of the indicator composition in a water sample. The pH of the indicator composition can be pH-controlled in a variety of ways, such as, through the selection and incorporation of one or more acidified and/or alkaline constituent components in the formulated indicator composition and/or through the incorporation of one or more pH regulating components in the composition that function to modify the pH of the resultant solution formed from the indicator composition. In some aspects, the indicator composition comprises at least one buffer. Suitable buffers for use in the indicator composition of the present disclosure may be effective to form a resultant solution achieving a target pH threshold and/or be within a target pH range. In some aspects, the buffer is able to maintain pH at a level less than about 7. For example, the pH of the resultant solution may be at least 0.5, such as at least 1.0, at least 1.5, at least 2.0, at least 2.5, at least 3.0, at least 3.5, at least 4.0, or at least 4.5. Additionally or alternatively, the pH of the resultant solution may be less than 7.0, such as less than 6.0, less than 5.0, less than 4.5, less than 4.0, less than 3.5, less than 3.0, or less than 2.5. In some examples, the pH of the resultant solution is within a range from about 1.0 to about 7.0, such as from about 1.5 to about 6.5, from about 2.0 to about 6.0, or from about 3.0 to about 6.0.

In some implementations, the buffer may comprise at least one of lactic acid, acetic acid, formic acid, uric acid, malic acid, tartaric acid, phthalic acid, citric acid, oxalic acid, phosphoric acid, MES (2-(N-Morpholino)ethanesulfonic acid), 4-Morpholineethanesulfonic acid, sulfamic acid, benzyl sulfonic acid, methylbenzyl sulfonic acid, glycine, and salts thereof. In some embodiments, the buffer comprises citric acid. In further embodiments, the buffer comprises citric acid and an alkali metal citrate. The buffer may be present within the indicator composition in an amount of from about 0.1 wt % to about 8 wt %, from about 0.1 wt % to about 7 wt %, from about 0.1 wt % to about 6 wt %, from about 0.1 wt % to about 5 wt %, or from about 0.2 wt % to about 4 wt %. In some implementations, the buffer may be present within the indicator composition in an amount of less than 8 wt %, less than 7 wt %, less than 6 wt %, or less than 5 wt %.

In some embodiments, the indicator composition includes a range extender comprising an alkali metal citrate and/or includes a buffer comprising citric acid and an alkali metal citrate. In some embodiments, the indicator composition consists essentially of the calcium indicator CPA III, the range extender an alkali metal citrate, the buffer citric acid and an alkali metal citrate, and water to form an indicator solution. In further embodiments, alternative calcium indicators, range extenders, and buffers may be used as described herein.

The indicator composition may optionally include a masking reagent. In some embodiments, the masking reagent preferentially binds magnesium. Representative magnesium masking reagents include 8-hydroxyquinoline, 8-hydroxyquinoline-5-sulfonic acid, and any combination thereof. In preferred embodiments, the indicator composition does not include a masking reagent, such as magnesium masking reagents. The indicator composition can further include optional additives. Optional additives may include one or more of a carrier, a biocide, a surfactant, and a filler and/or binding agent.

The indicator composition comprising the calcium indicator, range extender, and buffer may be provided directly to a water source or water sample. In some implementations, the indicator composition is provided as a solid composition. By the term “solid”, it is meant that the hardened composition will not flow and will substantially retain its shape under moderate stress or pressure or mere gravity. A solid may be in various forms such as a powder, a flake, a granule, a pellet, a tablet, a lozenge, a puck, a briquette, a brick, a solid block, a unit dose, or another solid form known to those of skill in the art.

In addition, the term “solid” refers to the state of the composition under the expected conditions of storage and use of the solid composition. Many of the current calcium indicators available are not thermally stable, i.e., may only be thermally stable in liquid form for about 2 weeks. However, the indicator compositions according to the disclosure can remain dimensionally stable under elevated temperature and humidity storage conditions for periods much longer than 2 weeks. For example, the indicator composition can remain dimensionally stable at a temperature of 50° C. (about 122° F.) and 70% relative humidity for a period of at least one month, such as at least two months, at least 6 months, or at least one year (e.g., a period from one month to one year). The term “dimensionally stable” means that the solid composition does not change size in any measured dimension by more than 1% when exposed to the noted environmental conditions outside of packaging protection for the subject period.

The indicator composition may take forms including, but not limited to: a cast solid product; an extruded, molded or formed solid pellet, block, tablet, powder, granule, flake; pressed solid; or the formed solid can thereafter be ground or formed into a powder, granule, or flake. In some implementations, the calcium indicator, range extender, buffer, and optional additives may be grinded together as a homogenous dry powder and pressed into a pressed solid form. In some implementations, the solid composition is provided as a cast solid, an extruded, molded, or formed solid, or a pressed solid comprising a pellet, block, tablet, powder, granule, or flake.

The indicator composition may be made by blending the dry constituent ingredients in appropriate ratios or agglomerating the materials in appropriate agglomeration systems. Pelletized materials can be manufactured by compressing the solid granular or agglomerated materials in appropriate pelletizing equipment to result in appropriately sized pelletized materials. Solid block and cast solid block materials can be made by introducing into a container either a pre-hardened block of material or a castable liquid that hardens into a solid block within a container. Example containers include disposable plastic containers or water-soluble film containers. Other suitable packaging for the composition includes flexible bags, packets, shrink wrap, and water-soluble film such as polyvinyl alcohol.

The indicator composition may be formed using a batch or continuous mixing system. In an example, a single- or twin-screw extruder is used to combine and mix one or more components at high shear to form a homogeneous mixture. In some embodiments, the processing temperature is at or below the melting temperature of the components. The processed mixture may be dispensed from the mixer by forming, casting or other suitable means, whereupon the composition hardens to a solid form. The structure of the matrix may be characterized according to its hardness, melting point, material distribution, crystal structure, and other like properties according to known methods in the art. Generally, a solid composition processed according to the disclosure is substantially homogeneous with regard to the distribution of ingredients throughout its mass and is dimensionally stable.

In an extrusion process, one or more liquid and/or solid components are introduced into a final mixing system and are continuously mixed until the components form a substantially homogeneous semi-solid mixture in which the components are distributed throughout its mass. The mixture is then discharged from the mixing system into, or through, a die or other shaping means. The product is then packaged.

In a casting process, one or more liquid and/or solid components are introduced into a final mixing system and are continuously mixed until the components form a substantially homogeneous liquid mixture in which the components are distributed throughout its mass. Once the mixing is complete, the product is transferred to a packaging container where solidification takes place.

In a pressed solid process, a flowable solid, such as granular solids, powders, or other particle solids are combined under pressure. In a pressed solid process, flowable solids of the compositions are placed into a form (e.g., a mold or container). The method can include gently pressing the flowable solid in the form to produce the indicator composition. Pressure may be applied by a block machine or a turntable press, or the like. Pressure may be applied at about 1 to about 2000 psi, about 1 to about 300 psi, about 5 psi to about 200 psi, or about 10 psi to about 100 psi. In certain embodiments, the methods can employ pressures as low as greater than or equal to about 1 psi, greater than or equal to about 2, greater than or equal to about 5 psi, or greater than or equal to about 10 psi. As used herein, the term “psi” or “pounds per square inch” refers to the actual pressure applied to the flowable solid being pressed and does not refer to the gauge or hydraulic pressure measured at a point in the apparatus doing the pressing. The method can include a curing step to produce the indicator composition. As referred to herein, an uncured composition including the flowable solid is compressed to provide sufficient surface contact between particles making up the flowable solid that the uncured composition will solidify into a stable composition. A sufficient quantity of particles (e.g., granules) in contact with one another provides binding of particles to one another effective for making a stable solid composition. Inclusion of a curing step may include allowing the pressed solid to solidify for a period of time, such as a few hours, or about 1 day (or longer). In additional aspects, the methods could include vibrating the flowable solid in the form or mold.

In some examples, the indicator composition is formed into a solid having a weight of at least 0.05 grams, at least 0.1 grams, at least 0.5 grams, at least 1 gram, at least 5 grams, or at least 10 grams, such as at least 100 grams, at least 1 kg, or at least 10 kg. For example, the composition may be formed into a solid having a mass from 1 to 100 kilograms, such as from 1 to 25 kilograms. In alternative examples, the composition may be formed into a solid having a mass from about 0.05 grams to about 100 grams, such as from about 0.1 grams to about 50 grams.

In some aspects, the solid compositions can provide for a stabilized source of functional materials. In further embodiments, the indicator composition may be dissolved, for example, in an aqueous medium, to create a concentrated reagent solution. The reagent solution may be directed to a storage reservoir for later use and/or dilution, or may be applied directly to a point of use. For example, an indicator composition may be dissolved in water to form a use solution. The use solution/reagent solution may then be added to a water sample of the water treatment system. Alternatively, the indicator composition may be provided as a solid composition and dissolved directly in a water sample of the water treatment system.

The indicator composition can be dissolved substantially completely in the water to which the composition is added. For example, the composition may exhibit about 100% solubility in the water in which it is added. The composition may dissolve in the water to which the composition is added over a time period of 1 hour or less, such as 30 minutes or less, 15 minutes or less, 10 minutes or less, 5 minutes or less, 2 minutes or less, 1 minute or less, 30 seconds or less, 20 seconds or less, 10 seconds or less, or 5 seconds or less. The water to which the solid composition is added may optionally be mixed to aid and accelerate dissolution. The temperature of the water to which the composition is added may vary and, in some examples, may be in a range from about 20° C. to about 80° C., such as from about 20° C. to about 30° C., from about 30° C. to about 40° C., from about 40° C. to about 50° C., from about 50° C. to about 60° C., from about 60° C. to about 70° C., or from about 70° C. to about 80° C.

In an application, a method of measuring calcium hardness is disclosed. The method may include extracting a sample of water containing calcium from a process water system, such as any process water system disclosed herein. In some aspects, the method includes adding an indicator composition, such as any of those disclosed herein, to the sample of water to form an optical analysis solution. In some embodiments, the indicator composition comprises a calcium indicator, a range extender, and a buffer, as disclosed herein. The method may further include optically analyzing the optical analysis solution and determining therefrom a concentration of calcium in the sample of water. Depending on the concentration of calcium in the sample of water, the addition of calcium control agents (such as, corrosion inhibitors) to the process water system can be controlled.

The indicator composition disclosed herein can be used in a process water system, such as, but not limited to, boiler water systems, wastewater treatment systems, and cooling systems (e.g., cooling towers, radiators, heat pipes, etc.). In additional aspects, the methods disclosed herein may be further used in desalination systems, pipes, drilling equipment (e.g., drill strings, drilling mud, etc.), tracking and fracking equipment, paper or pulp processing systems, water purification systems, ware washing, evaporators, condensers, filtration, mining, water softening, pumps, storage vessels, or any other systems using water sources or that contact one or more surfaces therein. During use, as the scale and/or corrosion promoting content of the water sources concentrate (e.g., through evaporation) and/or as thermal shock occurs, corrosive conditions intensify, corrosion initiates, and scale deposits on the surfaces of water containing systems from the water sources in contact therewith. Such surfaces may include the interior of pipes, storage vessels, radiators, heat pipes, filters, digesters, condensers, the exterior of cooling towers, or any other surface that contacts a water source. The surfaces of the water containing systems may include metals, plastics, glass, rubber or latex, fiberglass, concrete or stone, or any other material suitable to hold, transport, or filter water.

In some implementations, the source water in which the indicator composition is used is a cooling system including one or more of a water jacket, radiators, pipes, heat pipes, pumps, a cooling tower, etc. In some implementations, the indicator composition according to the disclosure can be used in an open circulating cooling water system, such as an open circulating cooling water system that includes one or more cooling towers that cool water via evaporative cooling. As the water source circulates through the water containing system and is evaporated on one or more portions thereof, the total dissolved solids content gradually concentrates with each cycle through the system. The corrosive/scaling materials therein, such as calcium ion, may reach a concentration where they begin to corrode and/or scale the surfaces in contact therewith, if left untreated. As calcium is one of the major ions that forms scale on a surface of water systems, an indicator composition of the present disclosure may be added to the process water source to measure the concentration/hardness of calcium in the water source. By accurately measuring the concentration of calcium within the water source, subsequent treatment steps may be adjusted in order to inhibit or prevent corrosion (e.g., pitting or oxidation) and/or scaling caused by the make-up (e.g., pH and/or dissolved solids) of the water source.

The process water source to be measured with the indicator composition as disclosed herein may include one or more corrodents/scale-forming agents therein, wherein the one or more corrodents/scale-forming agents comprises, consists essentially of, or consists of carbon dioxide, hydrogen sulfide, organosulfur compounds, metal cations, metal complexes such as aqueous metal cations, metal chelates and/or organometallic complexes, aluminum ions, ammonium ions, barium ions, chromium ions, cobalt ions, cuprous ions, cupric ions, calcium ions, ferrous ions, ferric ions, hydrogen ions, lead ions, magnesium ions, manganese ions, molybdenum ions, nickel ions, potassium ions, sodium ions, strontium ions, titanium ions, uranium ions, vanadium ions, zinc ions, bromide ions, carbonate ions, chlorate ions, chloride ions, chlorite ions, dithionate ions, fluoride ions, hypochlorite ions, iodide ions, nitrate ions, nitrite ions, oxide ions, perchlorate ions, peroxide ions, phosphate ions, phosphite ions, sulfate ions, sulfide ions, sulfite ions, hydrogen carbonate ions, hydrogen phosphate ions, hydrogen phosphite ions, hydrogen sulfate ions, hydrogen sulfite ions, carbonic acid, hydrochloric acid, nitric acid, sulfuric acid, nitrous acid, sulfurous acid, peroxy acids, phosphoric acid, ammonia, bromine, carbon dioxide, chlorine, chlorine dioxide, fluorine, hydrogen chloride, hydrogen sulfide, iodine, nitrogen dioxide, nitrogen monoxide, oxygen, ozone, sulfur dioxide, hydrogen peroxide, polysaccharide, or combinations thereof.

In some aspects, the process water source to be measured contains a corrodent/scale-forming agent comprising calcium ions. For example, the calcium ion present in the process water source may be from a source of at least one of calcium carbonate, calcium phosphate, and calcium sulfate. In some aspects, the source of calcium within the process water source comes from calcium-containing rocks and minerals, such as, but not limited to, limestone, marble, calcite, dolomite, gypsum, fluorite, and apatite.

Measuring calcium ions, especially in the fashion of real time, in the water source provides more insight as well as opportunities to control scale stress to the equipment in the process water. Various online methods of measuring calcium hardness are available, including, but not limited to, ion-selective electrode (ISE), fluorescence, titration, and colorimetric methods.

The ion-selective electrode (ISE) method uses a transducer that converts the activity of a specific ion dissolved in a solution into an electrical potential. The voltage is theoretically dependent on the logarithm of the ionic activity, according to the Nernst equation. The advantages of a calcium ISE probe include its smaller and compact body, linear response to calcium concentration up to a few thousands' ppm, and short response time. However, the need for frequent calibration of the calcium ISE probe in the field limits its application.

The use of fluorescence is an alternative method of measuring calcium. A few calcium reagents are available to complex the calcium ions in a water source and subsequently fluoresce. The fluorescence signal can then be used for the measurement of calcium as its intensity is proportional to the ion concentration of calcium. However, the fluorescence method is typically able to measure hardness at only a low concentration range. An automatic fluorescence method to measure magnesium, calcium, and total hardness for boiler water applications has been described in U.S. Pat. No. 8,956,875, which is hereby incorporated by reference in its entirety.

In alternative methods, the titration method for calcium measurement is well known and often applied in a lab setting. Some of the commercially available online calcium analyzers are based on a titration method by automating the titration procedure. In this method, the titrant is often the standard solution of ethylenediaminetetraacetic acid (EDTA). The calcium indicator can be any calcium complexing agent that changes colors when complexed with calcium ion, such as, for example, Eriochrome® Black T. In some aspects, if only calcium needs to be measured, potassium hydroxide will be needed to precipitate the magnesium ions prior to titration. For online analyzers, major challenges may include the complicated automation device and steps, the unclear endpoint, and interference from other ions to the indicator.

In further embodiments, a colorimeter method is employed. Beneficially, the colorimetric measurement is a convenient and inexpensive alternative to other methods of measuring calcium. A colorimeter is a device used to test the concentration of an analyte in a solution by measuring its absorbance of a specific wavelength of light. Only after the device has been calibrated can the device be used to find the densities and/or concentrations of the solutions. In certain aspects, optical filters are used in a colorimeter in order to eliminate any interference from the absorbance at unwanted wavelengths. In preferred implementations, a colorimeter method is employed to measure a concentration of calcium in a process water source.

The absorbance, transmittance, or reflectance at a certain wavelength or a wavelength range can be acquired by a spectrophotometer. A calibration curve with known calcium concentration is often obtained, and concentration of calcium is then calculated from the calibration curve. In some implementations, the methods of the present disclosure include optically analyzing a solution containing the process water to be analyzed and an indicator composition as disclosed herein (also described herein as the optical analysis solution). Using a spectrophotometer, an optical analysis solution is analyzed to measure at least one of the absorbance, transmittance, or reflectance of the optical analysis solution. In some embodiments, the optical analysis solution is optically analyzed to measure the absorbance of the optical analysis solution.

Once the absorbance, transmittance, or reflectance at a certain wavelength or a wavelength range is acquired from a sample solution, a linear calibration curve is formed. In certain embodiments, the calibration curve is developed based on the calcium reagent over a concentration of from 0 to about 1000 ppm of calcium as CaCO₃. In alternative embodiments, the calibration curve is developed based on a square root of the calcium concentration. In further embodiments, the calibration curve is developed based on the absorbance of the analyzed solution at a single wavelength. In alternative embodiments, the calibration curve is developed based on the absorbance of the analyzed solution at a ratio of two different wavelengths. In some aspects, the optical analysis is analyzed at one or more wavelengths. For example, the absorbance, transmittance, or reflectance of the optical analysis solution is analyzed at a wavelength within a range of from 300 nm to 800 nm, such as, from 350 nm to 750 nm, from 400 nm to 700 nm, or from 500 nm to 700 nm.

In further implementations, a correlation coefficient can be calculated from the calibration curve. In preferred embodiments, in order to provide suitable calibration curves for use in accurately measuring the calcium concentration in a water sample, the displayed correlation coefficient should be at least 0.99 with a quadratic regression. In certain aspects, the indicator compositions of the present disclosure provide a calibration curve with a correlation coefficient of 0.99 or greater.

With current calcium measuring methods, testing protocols are often ineffective for addressing high concentrations of analytes. Because many measurement protocols are optically based once a particular threshold is exceeded (such as up to and over 3 absorbance units) the reading is commonly “off the chart,” i.e., it is so dark that no further measurement can be achieved. This requires the addition of cumbersome dilution steps to the measurement process. As a result, the methods of the present disclosure incorporating the indicator composition described herein beneficially reduce the amount of free calcium to interact so that an accurate measurement of calcium can be obtained using the colorimetric method. In some aspects, the use of the indicator composition described herein result in an absorbance of 3 AU or less, 2 AU or less, or 1 AU or less. In some implementations, the indicator compositions described herein are employed to measure high concentrations of calcium (i.e., about 1 ppm to about 1400 ppm and higher) without additional dilutions.

In some embodiments, the amount of calcium in the water source or water sample may be present in a concentration range of at least about 1 ppm, such as, at least about 5 ppm, at least about 10 ppm, at least about 50 ppm, at least about 100 ppm, at least about 300 ppm, at least about 500 ppm, or at least about 1000 ppm. In further embodiments, the calcium may be present in the water source or water sample in a concentration range of from about 1 ppm to about 2000 ppm, from about 1 ppm to about 1500 ppm, from about 1 ppm to about 1400 ppm, from about 1 ppm to about 1000 ppm, from about 1 ppm to about 750 ppm, from about 1 ppm to about 500 ppm, from about 1 ppm to about 100 ppm, or from about 1 ppm to about 50 ppm. In further embodiments, the calcium may be present in the water source or water sample in a concentration range of from about 100 ppm to about 2000 ppm, from about 100 ppm to about 1500 ppm, from about 100 ppm to about 1000 ppm, from about 200 ppm to about 1000 ppm, or from about 250 ppm to about 1000 ppm.

In an application, the method of measuring calcium hardness further includes extracting a sample of water from the process water systems disclosed herein, comprising drawing a side stream from the process water system. The indicator composition can be added to the sample of water to form an optical analysis solution. The optical analysis solution can then be optically analyzed using an automated online analysis device. In some aspects, the online analysis device extracts the sample of water, and optically analyzes the optical analysis solution at least once per day. In further embodiments, the optical analysis solution is analyzed multiples times per day. In additional embodiments, the optical analysis solution is analyzed one or more times per week, or one or more times per month.

In some implementations, the addition of a calcium control agent to the process water system can be controlled or adjusted based on the determined concentration of calcium in the sample of water. In some aspects, the determined concentration of calcium is compared to at least one calcium threshold. Based on this comparison, a number of action steps may follow. In some aspects, if the concentration of calcium exceeds the threshold, at least one action step of the following may occur: (1) at least one calcium control agent is started; and (2) increasing the addition rate of the calcium control agent. In further aspects, if the concentration of calcium is less than the threshold, at least one action step of the following may occur: (1) stopping the addition of the calcium control agent; (2) decreasing the addition rate of the calcium control agent, and (3) keeping the amount of the calcium control agent the same.

In some examples, the indicator composition disclosed herein may be provided directly to a water source or water sample. In some implementations, the indicator composition is provided as a solid composition for dissolution directly to a source of water or water sample. The solid composition can be mixed with the source of water or water sample, e.g., by placing the solid in the source water and/or spraying the source water on the solid. In other examples, the indicator composition may be diluted with water to form a use solution prior to combining with a water source or water sample. The combination of the composition and source water may be mixed. Mixing may include one or more of batch wise, continuous, or incremental (e.g., supplemental, on-demand, or monitored) additions.

In some applications, the pH of the water source is between 7 and 14, such as between about 7 and about 10, between about 10 and 14, between about 9 and about 11, between about 7 and about 9, or between about 7 and about 8. In some other applications, the pH of the water source is between 0 and 7, such as between about 1 and about 6, between 5 and 6, between 4 and 5, between 3 and 4, between 2 and 3, or 1 and 2.

In further implementations, the indicator composition disclosed herein may be maintained in a closed system or an open system, augmented with additional water from outside the system (e.g., make-up water), and/or may be circulated out of the system (e.g., blow-down water) and replaced with additional water and/or indicator composition from outside the system. Such maintenance, augmentation, and removal of the water source and/or indicator composition allows a user to selectively control the concentration of composition in the water source and/or amount or rate of calcium control agent in the water containing system.

In an application, the system comprises an optical sensor configured to receive a sample of water containing an unknown concentration of calcium from a process water system. In some embodiments, the process water system may be an open circulating cooling water system, such as an open circulating cooling water system that includes one or more cooling towers that cool water via evaporative cooling. In other implementations, the process water system may be a wastewater treatment system, boiler water system, or other process water system.

The system can further comprise at least one pump fluidly coupled to an indicator composition as described herein. In some embodiments, the indicator composition comprises a calcium indicator, a range extender, and a buffer as described herein.

In some aspects, the system further comprises a controller that is communicatively coupled to both the optical sensor and at least one pump. In certain embodiments, the controller is configured to carry out a number of operational steps. The controller may be configured to control the pump to generate an optical analysis solution by at least combining the sample of water with the indicator composition. In further embodiments, the controller is configured to control the optical sensor to optically analyze the optical analysis solution. The optical analysis of the solution may comprise at least measuring an absorbance of the optical analysis solution.

In even further embodiments, the controller is configured to determine the concentration of calcium in the sample of water based on the optical analysis. In some aspects, the amount of calcium in the water source or sample of water may be present in a concentration range of at least about 1 ppm, such as, at least about 5 ppm, at least about 10 ppm, at least about 50 ppm, at least about 100 ppm, at least about 300 ppm, at least about 500 ppm, or at least about 1000 ppm. In further embodiments, the calcium may be present in the water source or water sample in a concentration range of from about 1 ppm to about 2000 ppm, from about 1 ppm to about 1500 ppm, from about 1 ppm to about 1400 ppm, from about 1 ppm to about 1000 ppm, from about 1 ppm to about 750 ppm, from about 1 ppm to about 500 ppm, from about 1 ppm to about 100 ppm, or from about 1 ppm to about 50 ppm. In further embodiments, the calcium may be present in the water source or water sample in a concentration range of from about 100 ppm to about 2000 ppm, from about 100 ppm to about 1500 ppm, from about 100 ppm to about 1000 ppm, from about 200 ppm to about 1000 ppm, or from about 250 ppm to about 1000 ppm.

The systems of the present disclosure may optionally comprise a calcium control agent pump. In some embodiments, the controller is communicatively coupled to the calcium control agent pump and configured to control the addition of a calcium control agent to the process water system based on the determined concentration of calcium in the sample of water. In some aspects, the determined concentration of calcium is compared to at least one calcium threshold. Based on this comparison, a number of action steps may follow. In some aspects, if the concentration of calcium exceeds the threshold, the controller is configured to implement at least one action step of the following: (1) at least one calcium control agent is started; and (2) increasing the addition rate of the calcium control agent. In further aspects, if the concentration of calcium is less than the threshold, the controller is configured to implement at least one action step of the following: (1) stopping the addition of the calcium control agent; (2) decreasing the addition rate of the calcium control agent, and (3) keeping the amount of the calcium control agent the same.

All publications and patent applications in this specification are indicative of the level of ordinary skill in the art to which this invention pertains. All publications and patent applications are herein incorporated by reference to the same extent as if each individual publication or patent application was specifically and individually indicated as incorporated by reference.

### EXAMPLES

Embodiments of the present invention are further defined in the following non- limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Calcium hardness in water was measured via a colorimetric method using various formulations. Table 1 illustrates a non-limiting example of a reagent mixture for measuring calcium within a water sample. The formula provided in Table 1 was prepared using chlorophosphonazo III (CPA III) as the calcium indicator. Sodium citrate was added to act as a weak ligand to compete with CPA III for calcium ions within the water sample. This allowed for a smaller amount of remaining calcium ions available to react with CPA III. Citric acid was further added to the formulation as a pH buffer to maintain the pH less than 7. The composition was subsequently diluted with water to form a reagent mixture.

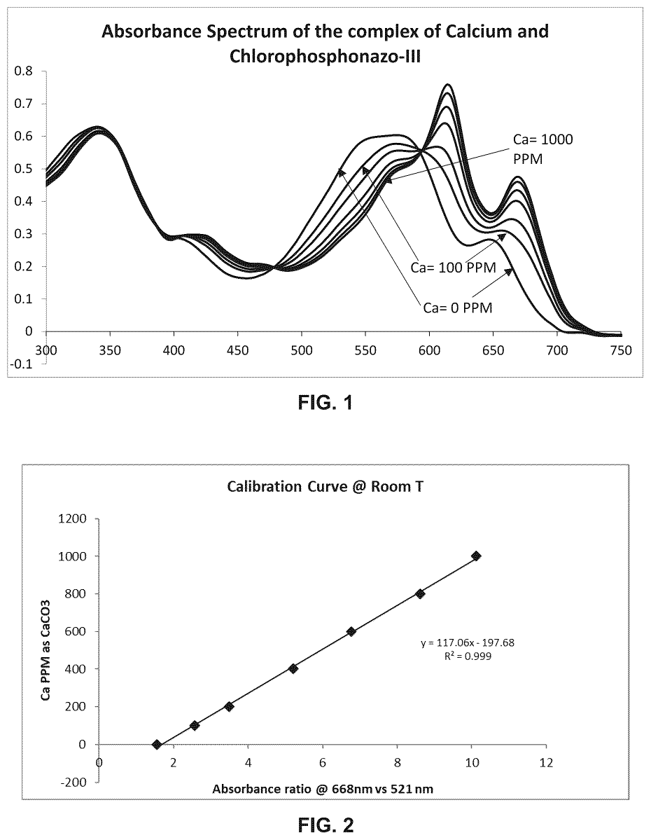

The reagent mixture from Table 1 was introduced to a water sample containing calcium. An absorbance spectrum provided by the complex of calcium within the water sample and CPA III of the reagent mixture was generated and shown in FIG. 1. A linear calibration curve for calcium between 0 ppm and 1000 ppm of CaCO₃ was formed with a correlation coefficient of 0.9990 as shown in FIG. 2. The calibration curve was formed with an absorbance ratio at a wavelength of 668 nm versus 521 nm as provided in FIG. 1.

TABLE 1

| | | Concentration |
|---|---|---|
| Component | Function | % (w/w) |
| Chlorophosphonazo III | Calcium indicator | 0.05 |
| Sodium citrate tribasic | Range extender | 4.71 |
| dihydrate | | |
| Citric acid | pH buffer | 2.31 |
| Water | | 92.93 |
| Total | | 100 |

Additional reagent formulations were prepared as shown in Table 2. The reagent formulations in Table 2 were prepared with varying concentrations of CPA III, citric acid, and sodium citrate.

TABLE 2

| | CPA III | Citric Acid | Sodium Citrate |
|---|---|---|---|
| Formula # | (g/100 mL) | (g/100 mL) | (g/100 mL) |
| S-7 | 0.2025 | 5.31 | 10.8 |
| S-12 | 0.045 | 2.36 | 4.8 |
| S-23 | 0.045 | 0.590 | 1.2 |
| S-24 | 0.0450 | 0.236 | 0.480 |
| S-25 | 0.045 | 0.442 | 0.900 |
| S-26 | 0.045 | 0.331 | 0.675 |
| D-1 | 0.09 | 2.36 | 4.8 |
| D-2 | 0.25 | 2.36 | 4.8 |
| D-3 | 0.10125 | 5.31 | 10.8 |
| D-4 | 0.045 | 3.6 | 7.2 |
| D-5 | 0.025 | 3.6 | 7.2 |

Linear calibration curves were further generated using the formulations as provided in Table 2 above under specified conditions of the water sample or solution. In various formulations provided in Table 3 below, the reagent mixture was further combined with the water sample or solution. The water sample or solution was prepared as follows:
(1) Prepare stock solution containing 10,000 ppm of calcium as CaCO₃: 14.70 grams of calcium chloride dihydrate (CaCl₂·2H₂O) powder was added to a 1.0-liter volumetric flask. Deionized (DI) water was added to the 1000 mL mark. The powder was completely dissolved.
(2) Prepare stock solution containing 10,000 ppm of magnesium as CaCO₃: 20.33 grams of magnesium chloride (MgCl₂·6H₂O) powder was added to a 1.0-liter volumetric flask. DI water was added to the 1000 mL mark. The powder was completely dissolved.
(3) Prepare the water sample or solution: The following stock solutions were blended in the indicated amounts in Table 3.

TABLE 3

| | Ca | Mg | Ca | Mg | | |
|---|---|---|---|---|---|---|
| Solution | (ppm as | (ppm as | stock | stock | | Total |
| # | CaCO3) | CaCO3) | (mL) | (mL) | DI Water | (mL) |
| 1 | 0 | 0 | 0 | 0 | Add to 1000 mark | 1000 |
| 2 | 100 | 50 | 10 | 5 | Add to 1000 mark | 1000 |
| 3 | 200 | 100 | 20 | 10 | Add to 1000 mark | 1000 |
| 4 | 400 | 200 | 40 | 20 | Add to 1000 mark | 1000 |
| 5 | 600 | 300 | 60 | 30 | Add to 1000 mark | 1000 |
| 6 | 800 | 400 | 80 | 40 | Add to 1000 mark | 1000 |
| 7 | 1000 | 500 | 100 | 50 | Add to 1000 Mark | 1000 |
| 8 | 1200 | 600 | 120 | 50 | Add to 1000 Mark | 1000 |
| 9 | 1400 | 700 | 140 | 50 | Add to 1000 Mark | 1000 |

The specified conditions of the water sample or solution and resulting correlation coefficients are provided Table 4. As shown in Table 4, the linearization of each specified formula was measured at either a ratio of two wavelengths or at a single wavelength. The linearization was further generated from either the calcium concentration as (CaCO₃), or as a square root of the calcium concentration. The correlation coefficient was further determined based on the calibration curve generated for each formula as provided within Table 4. A correlation coefficient of at least 0.99 is considered to provide sufficient accuracy. As the absorbance for both of Formula S-7 and D-3 hit the limit of detection, no results were available for analysis.

TABLE 4

| | | Linearization | Solution | |
|---|---|---|---|---|
| | Calcium as | (Absorbance at Specified | to | R² |
| Formula | CaCO3 | Wavelength versus | Reagent | (Correlation |
| # | (ppm) | Calcium Concentration) | Ratio | Coefficient) |
| S-12 | 0-1000 | 660/525 vs Ca Conc | 1:2 | 0.9447 |
| | | | 1:1 | 0.8638 |
| S-23 | 0-100 | 668/521 vs Ca Conc | 1:2 | 0.9962 |
| S-24 | 0-100 | 668/521 vs Ca Conc | 1:2 | 0.9900 |
| S-25 | 0-50 | 668/524 vs Ca Conc | 1:2 | 0.9993 |
| | 0-50 | 629 vs Ca conc | 1:2 | 0.9951 |
| | 0-50 | 620 vs Ca conc | 1:2 | 0.9973 |
| | 0-300 | 668/524 vs Ca Conc | 1:2 | 0.9126 |
| S-26 | 0-50 | 668/524 vs Ca Conc | 1:2 | 0.9997 |
| D-1 | 0-500 | 572 vs Ca Conc | 1:2 | 0.9984 |
| | 0-800 | 572 vs Ca Conc | 1:2 | 0.9883 |
| | 0-1000 | 572 vs Ca Conc | 1:2 | 0.9844 |
| | 0-1000 | 662vs SQRT(Ca Conc) | 1:2 | 0.9763 |
| D-2 | 0-1000 | 662vs SQRT(Ca Conc) | 1:2 | 0.9794 |
| D-4 | 0-1000 | 525 vs SQRT (Ca Conc) | 1:2 | 0.9978 |
| | 0-1000 | 660 vs SQRT (Ca Conc) | 1:2 | 0.9937 |
| | 0-1400 | 525 vs SQRT (Ca Conc) | 1:2 | 0.9980 |
| | 0-1400 | 660 vs SQRT (Ca Conc) | 1:2 | 0.9814 |
| | 0-2000 | 525 vs SQRT (Ca Conc) | 1:2 | 0.9854 |
| | 0-2000 | 660 vs SQRT (Ca Conc) | 1:2 | 0.9741 |
| D-5 | 0-300 | 660 vs SQRT (Ca Conc) | 1:2 | 0.9968 |
| | 0-1000 | 660 vs SQRT (Ca Conc) | 1:2 | 0.9989 |
| | | and others | | |
| | 0-1400 | 660 vs SQRT (Ca Conc) | 1:2 | 0.9948 |
| | 0-1400 | 660 vs SQRT (Ca Conc) | 1:1.8 | 0.9912 |
| | | | 1:2 | 0.9948 |
| | | | 1:2.2 | 0.9936 |

Further reagent compositions were evaluated to identify linearity. The formulation and results are shown in Table 5. The data in Table 5 is based on the ratio of absorbance at 668 nm to absorbance at 521 nm. The results are based on either the ratio as is, or as an exponential function of the ratio of absorbance at 668 nm to absorbance at 521 nm. In embodiments where the exponential function of the ratio was measured, the concentration of calcium was plotted against the exponential function of the ratio of the absorbance at 668 nm to absorbance at 521 nm.

TABLE 5

| | | | Sodium | Solution | Reagent | |
|---|---|---|---|---|---|---|
| | CPA III | Citric Acid | Citrate | volume | volume | |
| Formula | (g/100 mL) | (g/100 mL) | (g/100 mL) | (mL) | (mL) | Comments |
| 1 | 0.09 | 0.38 | 10.0 | 1 | 9 | Non-linear |
| 2 | 0.09 | 9.6 | 14.7 | 1 | 1 | Near linear with |
| | | | | | | exp ratio |
| 3 | 0.12 | 4.8 | 7.41 | 1 | 9 | Non-linear |
| 4 | 0.045 | 1.18 | 2.4 | 1 | 9 | Linear up to 1000 |
| | | | | | | ppm if exp ratio |
| 5 | 0.095 | 1.30 | 2.5 | 1 | 9 | Linear up to 1000 |
| | | | | | | if exp ratio |
| 6 | — | — | — | — | — | — |
| 7 | 0.09 | 2.36 | 4.8 | 4 | 9 | Linear up to 800 |
| | | | | | | ppm of Ca |
| 8 | 0.045 | 1.77 | 3.6 | 1 | 2 | Linear up to 800 |
| | | | | | | ppm of Ca |
| 9 | 0.045 | 2.36 | 4.8 | 1 | 2 | Linear up to 1400 |
| | | | | | | ppm if exp ratio |
| 10 | 0.061 | 4.72 | 9.6 | 1 | 2 | Linear up to 1400 |
| | | | | | | ppm Ca |
| 11 | 0.061 | 9.45 | 9.6 | 1 | 2 | Linear up to 1400 |
| | | | | | | ppm Ca |
| 12 | 0.045 | 2.36 | 3.61 | 1 | 2 | Non-linear |
| 13 | 0.27 | 10.63 | 21.6 | 1 | 1 | Non-linear |

### Example 2

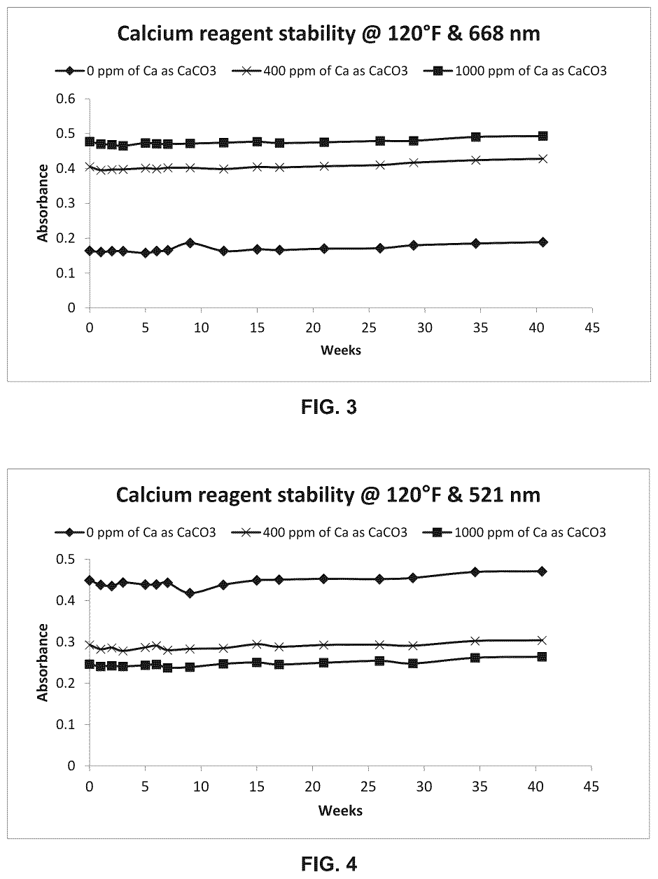

The thermal stability of the indicator composition of the present disclosure was further evaluated. In a non-limiting example, the thermal stability of the reagent formulation as provided in Table 1 was evaluated at two different wavelengths and three different calcium concentrations (as CaCO₃) Stability was observed at both a wavelength of 668 nm as shown in FIG. 3, as well as at a wavelength of 521 nm as shown in FIG. 4. The stability was measured for calcium concentrations at 0 ppm, 400 ppm, and 1000 ppm, and observed at a temperature of 120° F. over a period of 40 weeks. As can be seen from FIG. 3 and FIG. 4, the absorbance reading remained stable throughout the 40-week period. As such, the formulations of the present disclosure beneficially provide for thermally stable compositions.

Various examples have been described. These and other examples are within the scope of the following claims.

What is claimed is:

1. A method of measuring calcium hardness comprising:
extracting a sample of water containing calcium from a process water system;
adding an indicator composition to the sample of water to form an optical analysis solution, the indicator composition comprising a calcium indicator, a range extender, and a buffer;
optically analyzing the optical analysis solution and determining therefrom a concentration of the calcium in the sample of water; and
controlling addition of a calcium control agent to the process water system based on the determined concentration of calcium in the sample of water.

2. The method of claim 1, wherein the calcium indicator comprises chlorophosphonazo III or chlorophosphonazo I.

3. The method of claim 1, wherein the range extender comprises at least one of a polycarboxylic acid, an acrylate-based polymer, a polymaleic anhydride (PMA), a poly epoxy succinic acid (PESA), a maleic anhydride and sulfonated styrene copolymer (MA/SS), a polyaspartic acid, a chelating agent, and salts thereof.

4. The method of claim 1, wherein the range extender comprises an alkali metal citrate.

5. The method of claim 1, wherein the range extender is (i) a polymer selected from the group consisting of a polyacrylate (PAA), a polymaleic anhydride (PMA), a poly epoxy succinic acid (PESA), a poly(acrylate-co-acrylamide) copolymer (AA/AM), an acrylic acid and hydroxypropylacrylate copolymer (AA/HPA), an acrylic acid and 2-acrylamido-2-methyl propane sulfonate copolymer (AA/AMPS), a maleic anhydride and sulfonated styrene copolymer (MA/SS), an acrylic acid/acrylamide/tertiary butyl acrylamide copolymer (AA/AM/t-BAM), an acrylic acid/2-acrylamido-2-methyl propane sulfonate/tertiary butyl acrylamide (AA/AMPS/t-BAM), an acrylic acid/sulfonated styrene/2-acrylamido-2-methyl propane sulfonate (AA/SS/AMPS), an acrylic acid/acrylamide/aminomethyl sulfonate copolymer(AA/AM/AMS), polyaspartic acid, and combinations thereof; or (ii) a chelating agent selected from the group consisting of L-glutamic acid N—N-diacetic acid tetrasodium salt (GLDA), iminodisuccinic acid ((N-1,2-dicarboxyethyl)-D,L-aspartate acid), ethylenediamine-N,N′-disuccinic acid (EDDS), methylglycinediacetic acid (MGDA), ethyleneglycol-O,O′-bis(2-aminoethyl)-N,N, N′,N′-tetraacetic acid (EGTA), ethylenediamine-tetraacetic acid disodium salt (EDTA), ODS (oxygen disuccinic acid), salts thereof, and combinations thereof.

6. The method of claim 1, wherein the buffer is at least one of lactic acid, acetic acid, formic acid, uric acid, malic acid, tartaric acid, phthalic acid, citric acid, oxalic acid, phosphoric acid, MES (2-(N-Morpholino)ethanesulfonic acid), 4-Morpholineethanesulfonic acid, sulfamic acid, benzyl sulfonic acid, methylbenzyl sulfonic acid, glycine, salts thereof, or a combination thereof.

7. The method of claim 1, wherein the buffer comprises citric acid and an alkali metal citrate.

8. The method of claim 1, wherein the indicator composition is provided as a solid composition comprising a cast solid, an extruded, molded, or formed solid, or a pressed solid comprising a pellet, block, tablet, powder, granule, or flake.

9. The method of claim 1, wherein the indicator composition is diluted with water to form a use solution.

10. The method of claim 1, wherein the optical analysis solution has a pH of less than 7.0.

11. The method of claim 1, wherein the indicator composition consists essentially of the calcium indicator chlorophosphonazo III, the range extender an alkali metal citrate, the buffer citric acid and an alkali metal citrate, and water to form an indicator solution, and wherein the indicator solution has a pH less than 7.0.

12. The method of claim 1, wherein:
the calcium indicator comprises less than about 0.5 wt % of the indicator composition;
the range extender ranges from about 0.1 wt % to about 10 wt % of the indicator composition; and
the buffer ranges from about 0.1 wt % to about 5 wt % of the indicator composition.

13. The method of claim 1, wherein a concentration of calcium in the sample of water ranges from about 1 ppm to about 1000 ppm.

14. The method of claim 1, wherein the process water system comprises at least one of a boiler water system, a wastewater system, and a cooling water system, the cooling water system comprising a cooling tower that reduces a temperature of a cooling water stream through evaporative cooling.

15. The method of claim 1, wherein optically analyzing the optical analysis solution comprises measuring at least one of absorbance and transmittance of the optical analysis solution.

16. The method of claim 1, wherein optically analyzing the optical analysis solution comprises optically analyzing the solution at one or more wavelengths within a range from 500 nm to 700 nm.

17. The method of claim 1, wherein:
extracting the sample of water comprises drawing a side stream from the process water system, and
adding the indicator composition to the sample of water and optically analyzing the optical analysis solution comprises adding the indicator composition to the sample of water and optically analyzing the optical analysis solution using an automated online analysis device.

18. The method of claim 17, wherein the automated online analysis device extracts the sample of water, adds the indicator composition, and optically analyzes the optical analysis solution at least once per day.

19. The method of claim 18, wherein controlling addition of a calcium control agent to the process water system based on the determined concentration of calcium in the sample of water comprises comparing the determined concentration of calcium to at least one calcium threshold, and
at least one of starting addition of the calcium control agent and increasing an addition rate of the calcium control agent if the determined concentration of calcium exceeds the threshold, and
at least one of stopping addition of the calcium control agent, decreasing the addition rate of the calcium control agent, or keeping the amount of the calcium control agent the same if the determined concentration of calcium is less than the threshold.

20. A system comprising:
an optical sensor configured to receive a sample of water containing an unknown concentration of calcium from a process water system;
an indicator composition comprising a calcium indicator, a range extender, and a buffer;
at least one pump fluidly coupled to the indicator composition; and
a controller communicatively coupled to the optical sensor and the pump, wherein the controller is configured to:
control the pump to generate an optical analysis solution by at least combining the sample of water with the indicator composition;
control the optical sensor to optically analyze the optical analysis solution; and
determine a concentration of calcium in the sample of water based on the optical analysis.

21. The system of claim 20, wherein the system further comprises a calcium control agent pump, wherein the controller is communicatively coupled to the calcium control agent pump and configured to control addition of a calcium control agent to the process water system based on the determined concentration of calcium in the sample of water.

22. The system of claim 21, wherein the controller is configured to control addition of the calcium agent to the process water system based on the determined concentration of calcium in the sample of water by at least comparing the determined concentration of calcium to at least one calcium threshold, and
at least one of starting addition of the calcium control agent and increasing an addition rate of the calcium control agent if the determined concentration of calcium exceeds the threshold, and
at least one of stopping addition of the calcium control agent, decreasing the addition rate of the calcium control agent, or keeping the amount of calcium control agent the same if the determined concentration of calcium is less than the threshold.

23. The system of claim 20, wherein:
the calcium indicator comprises chlorophosphonazo III or chlorophosphonazo I;
the range extender comprises at least one of a polycarboxylic acid, an acrylate-based polymer, a polymaleic anhydride (PMA), a poly epoxy succinic acid (PESA), a maleic anhydride and sulfonated styrene copolymer (MA/SS), a polyaspartic acid, a chelating agent, and salts thereof; and
the buffer is at least one of lactic acid, acetic acid, formic acid, uric acid, malic acid, tartaric acid, phthalic acid, citric acid, oxalic acid, phosphoric acid, MES (2-(N-Morpholino) ethanesulfonic acid), 4-Morpholineethanesulfonic acid, sulfamic acid, benzyl sulfonic acid, methylbenzyl sulfonic acid, glycine, and salts thereof.

24. An indicator composition for measuring a concentration of calcium, the indicator composition comprising:
a calcium indicator;
a range extender; and
a buffer,
wherein the indicator composition has a use pH less than 7.0.

25. The indicator composition of claim 24, wherein the calcium indicator comprises chlorophosphonazo III or chlorophosphonazo I, wherein the range extender comprises at least one of a polycarboxylic acid, an acrylate-based polymer, a polymaleic anhydride (PMA), a poly epoxy succinic acid (PESA), a maleic anhydride and sulfonated styrene copolymer (MA/SS), a polyaspartic acid, a chelating agent, salts thereof, or combinations thereof, and wherein the buffer is at least one of lactic acid, acetic acid, formic acid, uric acid, malic acid, tartaric acid, phthalic acid, citric acid, oxalic acid, phosphoric acid, MES (2-(N-Morpholino) ethanesulfonic acid), 4-Morpholineethanesulfonic acid, sulfamic acid, benzyl sulfonic acid, methylbenzyl sulfonic acid, glycine, salts thereof, or combinations thereof.
